(19) United States
(12) Patent Application Publication
(10) Pub. No.: US 2025/0264652 A1
(43) Pub. Date: Aug. 21, 2025

(54) OPTICAL DIFFUSION DEVICE

(71) Applicant: FURUKAWA ELECTRIC CO., LTD., Tokyo (JP)

(72) Inventors: Hideaki HASEGAWA, Tokyo (JP); Junichi HASEGAWA, Tokyo (JP)

(21) Appl. No.: 19/200,692

(22) Filed: May 7, 2025

Related U.S. Application Data
(63) Continuation of application No. PCT/JP2023/039803, filed on Nov. 6, 2023.

(30) Foreign Application Priority Data
Nov. 11, 2022 (JP) 2022-180691

(51) Int. Cl. F21V 8/00; A61N 5/06

(52) U.S. Cl. CPC G02B 6/0008; A61N 5/0603; A61N 5/062; A61N 2005/063; A61N 2005/0666

(57) ABSTRACT

An optical diffusion device for photoimmunotherapy or photodynamic therapy comprises: a light transmission cable which transmits light emitted from a laser oscillator and which outputs the transmitted light from an output surface of a tip end part; a reflection member which has a refraction surface that refracts light output from the output surface; and a resin tube-shaped member into which the light transmission cable and the reflection member are inserted. The refraction surface is disposed in the tube-shaped member at a position which is a prescribed distance from the output surface, is inclined with respect to the axial direction X of the light transmission cable, causes laser light output from the output surface to be inclined by a prescribed angle or greater with respect to the axial direction of the light transmission cable, and outputs the laser light.

This application is based on and claims the benefit of priority to Japanese Patent Application No. 2022-180691 and 2022-180692 filed on Nov. 11, 2022 and is a Continuation Application of PCT Application No. PCT/JP2023/039803 filed on Nov. 6, 2023. The entire contents of each application are hereby incorporated herein by reference.

### TECHNICAL FIELD

The present invention relates to a light diffusion device for photoimmunotherapy or photodynamic therapy.

### BACKGROUND ART

As a conventional light diffusion device, there is known a device which includes an optical transmission cable including an optical transmission path through which light emitted from a light source is transmitted, and a lens provided at a distal end part of the optical transmission cable, and which irradiates light emitted from the optical transmission cable in a predetermined direction through the lens (for example, see Patent Document 1). A light diffusion device is used, for example, in photoimmunotherapy and photodynamic therapy performed as a therapy for cancer or the like, by inserting the distal end side of an optical transmission cable into a human body and irradiating laser light onto a drug that has been administered to the human body and reached cancer cells.

### CITATION LIST

### Patent Document

Patent Document 1: Japanese Patent No. 4659137

### DISCLOSURE OF THE INVENTION

### Problems to be Solved by the Invention

In photoimmunotherapy, it is necessary to irradiate light in a state in which the distal end side of the optical transmission cable is inserted into the human body or positioned in the vicinity of the tumor surface. In the case of organs such as the intestine and the esophagus, cancer cells to be irradiated are often present on the side surface of the organ, and it is important to efficiently irradiate light obliquely with respect to the axial direction of the optical transmission cable. In light diffusion devices, a part where light is refracted and the distal end side of an optical transmission cable are often covered with a member made of metal or quartz and fixed. However, in photoimmunotherapy or photodynamic therapy, since a treatment site is limited by an angle of emitted light, a material configuration having a higher degree of freedom with respect to a demand of a user of a device, such as biocompatibility, cost, and operability, is required for a configuration for irradiating light to a site to be irradiated.

It is an object of the present invention to provide a light diffusion device capable of irradiating light in a direction inclined with respect to the axial direction of an optical transmission cable, and having an excellent degree of freedom in selecting a material with respect to the needs of a user of the device in terms of biocompatibility, cost, operability, etc.

### Means for Solving the Problems

(1) A light diffusion device for photoimmunotherapy or photodynamic therapy includes an optical transmission cable configured to transmit light emitted from a light source and emit the transmitted light from an emission surface of a distal end part, a reflective member having a refractive surface that refracts the light emitted from the emission surface, and a tubular member made of resin into which the optical transmission cable and the reflective member are inserted. The refractive surface is disposed at a predetermined distance from the emission surface in the tubular member and is inclined with respect to an axial direction of the optical transmission cable. The light emitted from the emission surface is emitted in a direction that is inclined at a predetermined angle or more with respect to the axial direction of the optical transmission cable.
(2) In the light diffusion device according to (1), the reflective member is a rod-shaped member made of quartz or silicon and is spaced apart from the optical transmission cable in the tubular member. The refractive surface is formed on an end part of the rod-shaped member on a side adjacent to the optical transmission cable.
(3) In the light diffusion device according to (1) or (2), the refractive surface includes a vapor-deposited metal.
(4) In the light diffusion device according to any one of (1) to (3), the optical transmission cable is a plastic fiber including a core having an outer diameter of 500 μm or more, and a cladding made of resin and formed on an outer periphery of the core. An outer diameter of the refractive surface viewed from the axial direction of the optical transmission cable is larger than the outer diameter of the core.
(5) In the light diffusion device according to any one of (1) to (4), an unevenness of the refractive surface on which the light is incident is equal to or less than a wavelength of the light generated from the light source.
(6) In the light diffusion device according to any one of (1) to (5), the emission surface of the optical transmission cable is inclined with respect to the axial direction of the optical transmission cable.
(7) In the light diffusion device according to (6), the emission surface is inclined with respect to the axial direction of the optical transmission cable so as to face the refractive surface substantially in parallel.
(8) In the light diffusion device according to any one of (1) to (7), the refractive surface is formed in a curved surface shape that is concave with respect to the emission surface.

### Effects of the Invention

According to the present invention, it is possible to provide a light diffusion device capable of irradiating light in a direction inclined with respect to the axial direction of an optical transmission cable, and having an excellent degree of freedom in selecting a material with respect to the needs of a user of the device in terms of biocompatibility, cost, operability, etc.

### BRIEF DESCRIPTION OF THE DRAWINGS

FIG. 1 is a side view schematically showing an appearance of a light diffusion device according to a first embodiment of the present invention;

FIG. 2 schematically shows a light diffusion device according to the first embodiment of the present invention, and is a side view of the light diffusion device mainly irradiating laser light to the side;

FIG. 3 schematically shows a light diffusion device according to the first embodiment of the present invention, and is a side view of the light diffusion device that mainly irradiates laser light rearward;

FIG. 4 is a side view schematically showing a light diffusion device according to a second embodiment of the present invention;

FIG. 5 is a side view schematically showing a light diffusion device according to a third embodiment of the present invention;

FIG. 6 is a side view schematically showing a light diffusion device according to a fourth embodiment of the present invention;

FIG. 7 is a side view schematically showing an appearance of a light diffusion device according to a fifth embodiment of the present invention; and

FIG. 8 is a side view schematically showing the light diffusion device according to the fifth embodiment of the present invention.

### PREFERRED MODE FOR CARRYING OUT THE INVENTION

Hereinafter, embodiments of the present invention will be described with reference to the drawings. The present invention is not limited to the following embodiments. The drawings referred to in the following description merely schematically show shapes, sizes, and positional relationships so that the contents of the present disclosure can be understood. That is, the present invention is not limited to only the shapes, sizes, and positional relationships illustrated in the drawings.

### First Embodiment

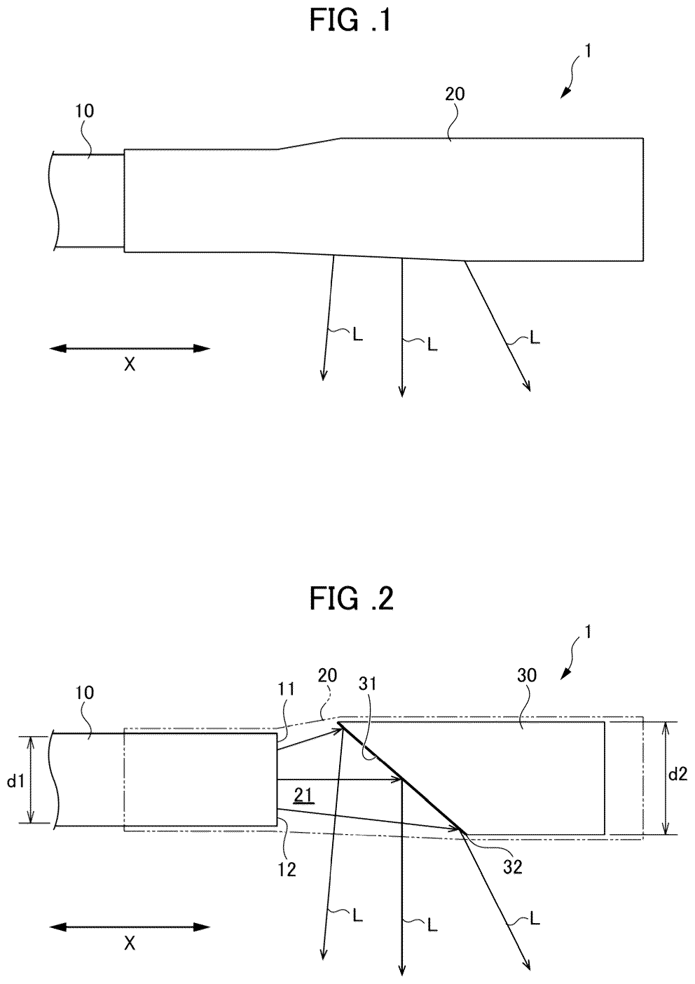

A light diffusion device 1 according to a first embodiment of the present invention will be described with reference to FIGS. 1 and 2. FIGS. 1 and 2 are side views of the light diffusion device 1. FIG. 1 shows the appearance of the distal end part side of the light diffusion device 1. FIG. 2 is a side view of the distal end part side of the light diffusion device 1 showing the structure in a tubular member 20. In FIG. 2, the tubular member 20 is indicated by a two-dot chain line.

The light diffusion device 1 of the present embodiment is mounted on a medical device for performing photoimmunotherapy, which is a method for treating cancer. The photoimmunotherapy treats cancer by administering to a human body a drug composed of an antibody that binds to cancer cells and a substance that reacts to light, and then irradiating the drug bound to the cancer cells with laser light L to destroy the cancer cells. The light diffusion device 1 is inserted into, for example, a conduit provided in an endoscope, and is used in a state where the distal end part thereof is exposed to the outside. The present invention is not limited to photoimmunotherapy, and can be used for photodynamic therapy.

As shown in FIGS. 1 and 2, the light diffusion device 1 includes a laser oscillator (not shown) as a light source, an optical transmission cable 10, a tubular member 20, and a rod-shaped member 30 as a reflective member.

The laser oscillator includes a semiconductor laser, generates laser oscillation by energizing the semiconductor laser, and generates laser light L. The laser oscillator generates red laser light L having a wavelength of 600 nm or more and 700 nm or less. The laser light L generated from the laser oscillator is a continuous wave.

The optical transmission cable 10 is an optical fiber cable having an optical transmission path through which the laser light L emitted from the laser oscillator is transmitted. The laser oscillator is disposed on the proximal end part side of the optical transmission cable 10, and the rod-shaped member 30 is provided on a distal end part 11 side. The optical transmission cable 10 transmits the laser light L generated in the laser oscillator through the optical transmission path and emits the laser light L from an emission surface 12 at the distal end part 11 toward the rod-shaped member 30. The emission surface 12 of the present embodiment is a surface perpendicular to the axial direction X of the optical transmission cable 10.

The optical transmission cable 10 according to the present embodiment is a plastic fiber, and includes a core (not shown) and a cladding (not shown) made of resin, formed on the outer periphery of the core. Examples of the resin forming the cladding include PTFE and PVDF. The outer diameter d1 of the core of the optical transmission cable 10 is preferably 500 μm or more. In the present embodiment, the outer diameter of the core is 500 μm. In the present embodiment, the emission surface 12 of the optical transmission cable 10 is a surface of the core at the distal end part 11. Preferably, the core has an outer diameter dimension corresponding to a multimode fiber. The optical transmission cable 10 of the present embodiment is a multimode fiber, and emits laser light L from a plurality of locations of the emission surface 12 as shown in FIG. 2. The optical transmission cable 10 of the present embodiment is a single-core optical fiber, but may be a multi-core optical fiber. The shape of the core may be an ellipse or a rectangle other than a perfect circle when viewed from the axial direction X of the optical transmission cable 10. The optical transmission cable 10 may be an optical fiber made of a quartz-based material.

The tubular member 20 is a cylindrical tube made of resin. The term “tube made of resin” herein includes both tubes made of resin only and tubes made mainly of resin. The tubular member 20 houses a part of the optical transmission cable 10 and the rod-shaped member 30 therein. The tubular member 20 is configured to be reducible in diameter. In the present embodiment, the optical transmission cable 10 is inserted into the tubular member 20 such that at least the distal end part 11 side is located inside the tubular member 20. As shown in FIG. 1, the optical transmission cable 10 is housed in the tubular member 20 in a state of extending in the axial direction of the tubular member 20. The resin forming the tubular member 20 preferably has a light transmittance of 50% or more. Examples of the resin forming the tubular member 20 include polyimide, FEP (tetrafluoroethylene-hexafluoropropylene copolymer), and acrylic resin.

The rod-shaped member 30 is made of quartz and is housed in the tubular member 20. The term “rod-shaped member 30 made of quartz” herein includes both a rod-shaped member 30 made of quartz only and a rod-shaped member 30 mainly made of quartz. Specifically, the rod-shaped member 30 is housed in the tubular member 20, extending in the axial direction of the tubular member 20 and spaced apart from the optical transmission cable 10. In the present embodiment, the rod-shaped member 30 is disposed substantially coaxially with the optical transmission cable 10 in the tubular member 20. The rod-shaped member 30 is entirely housed in the tubular member 20 and is not exposed to the outside. The optical transmission cable 10 and the rod-shaped member 30 are fixed in the tubular member 20 by, for example, setting the outer diameters thereof to be larger than the inner diameter of the tubular member 20 and tightening them with a force directed radially inward by the tubular member 20 (in a so-called interference fit state). The rod-shaped member 30 may be made of silicon. The term “rod-shaped member 30 made of silicon” herein includes both a rod-shaped member 30 made of silicon only and a rod-shaped member 30 mainly made of silicon.

A refractive surface 31 is formed at an end part of the rod-shaped member 30 on the optical transmission cable 10 side. The refractive surface 31 is an inclined surface made of quartz, formed by cutting the rod-shaped member 30 obliquely with respect to the axial direction thereof. The term “refractive surface 31 made of quartz” herein includes both a refractive surface 31 made of quartz only and a refractive surface 31 mainly made of quartz. The refractive surface 31 faces the emission surface 12 in the tubular member 20 and is disposed so as to be inclined with respect to the axial direction X of the optical transmission cable 10. The refractive surface 31 may be made of silicon. The term “refractive surface 31 made of silicon” herein includes both a refractive surface 31 made of silicon only and a refractive surface 31 mainly made of silicon.

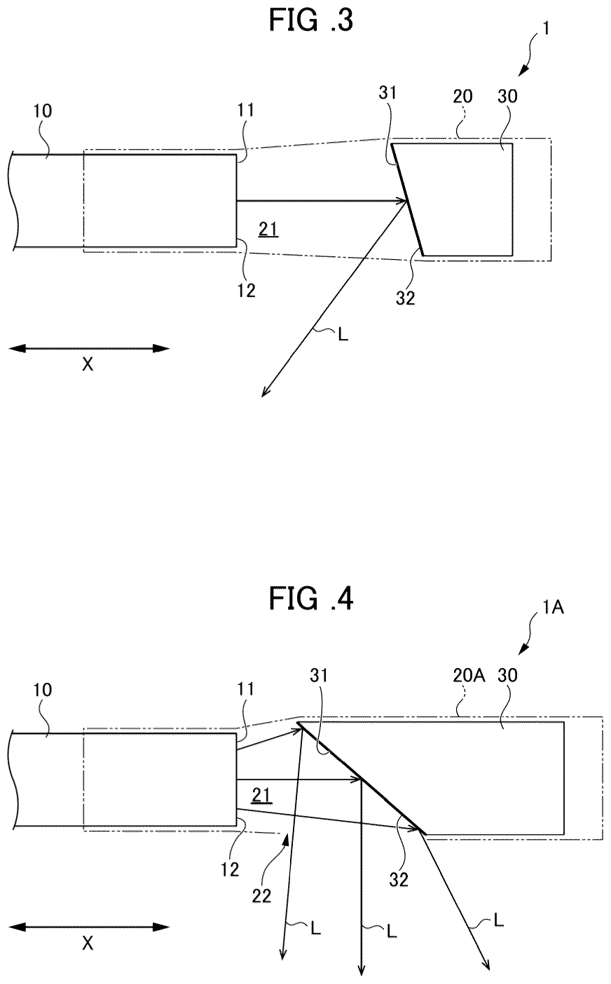

As shown in FIG. 2, the refractive surface 31 emits the laser light L emitted from the emission surface 12 at the distal end part 11 of the optical transmission cable 10 to the outside of the tubular member 20 in a direction that is inclined at a predetermined angle or more with respect to the axial direction X of the optical transmission cable 10. For example, as shown in FIG. 2, the refractive surface 31 refracts each laser light L emitted in the axial direction X of the optical transmission cable 10 from a plurality of locations of the emission surface 12, and emits the laser light L to the side of the tubular member 20. For example, the laser light L refracted through the refractive surface 31 passes through the tubular member 20, is emitted in a direction inclined with respect to the insertion direction of the optical transmission cable 10, and is irradiated onto cancer cells or the like present on the surface of an organ. Alternatively, for example, as shown in FIG. 3, the inclination of the refractive surface 31 may be set to be closer to the vertical direction with respect to the axial direction X of the optical transmission cable 10 than the refractive surface 31 shown in FIG. 2. With this configuration, as shown in FIG. 3, the laser light L can be irradiated rearward from the refractive surface 31. The axial direction X of the optical transmission cable 10 in the present specification means the axial direction of the optical transmission cable 10 at the distal end part 11.

As shown in FIG. 2, the refractive surface 31 of the present embodiment is formed in a planar shape as a whole. The unevenness of the refractive surface 31 on which the laser light L is incident is preferably equal to or less than the wavelength of the laser light L generated from the laser oscillator. For example, by mirror-polishing the refractive surface 31, it is possible to realize an unevenness equal to or less than the wavelength of the laser light L. A metal 32 is vapor-deposited on the refractive surface 31 of the present embodiment. Examples of the metal 32 vapor-deposited on the refractive surface 31 include gold, silver, and aluminum.

As shown in FIG. 2, the outer diameter d2 of the rod-shaped member 30 is larger than the outer diameter d1 of the core of the optical transmission cable 10. That is, the outer diameter of the refractive surface 31 viewed from the axial direction X of the optical transmission cable 10 is larger than the outer diameter d1 of the core. With this configuration, since the refractive surface 31 that receives the laser light L emitted from the optical transmission cable 10 is larger than the emission surface 12, it is possible to allow for misalignment of the position of the refractive surface 31 with respect to the optical transmission cable 10.

The refractive surface 31 is disposed at a predetermined distance from the emission surface 12 in the tubular member 20. The distance between the emission surface 12 and the refractive surface 31 is preferably in the range of 0.5 mm to 1 mm. A medium having a refractive index different from those of the emission surface 12 and the refractive surface 31 exists between the emission surface 12 and the refractive surface 31. For example, in the present embodiment, only space 21 exists as the medium having a different refractive index between the emission surface 12 and the refractive surface 31. A lens or the like having a refractive index different from those of the emission surface 12 and the refractive surface 31 and being in contact with both the emission surface 12 and the refractive surface 31 may be interposed between the emission surface 12 and the refractive surface 31 so as to fill the space 21.

Here, in photoimmunotherapy and photodynamic therapy, since laser light having an output of about 0.5 W to 2.0 W is used, the amount of heat generated in the tubular member 20 through which the laser light L from the optical transmission cable 10 passes is relatively small. Therefore, the heat resistance required for the member is relatively low, and as the material of the tubular member 20, a material made of a resin more excellent in biocompatibility can be used instead of metal, quartz, or the like. In photoimmunotherapy and photodynamic therapy, the optical transmission cable 10 that is a multimode fiber having a relatively large outer diameter d1 of the core of about 500 μm is mainly used. Thus, even if heat sufficient to deform the resin is applied to the tubular member 20, causing a shift of several μm in the relative position between the emission surface 12 and the refractive surface 13, optical effects resulting from the shift in the relative position is unlikely to occur. Therefore, the light diffusion device 1 according to the present embodiment uses the tubular member 20 made of resin suitable for use in photoimmunotherapy or photodynamic therapy.

### Second Embodiment

Next, a light diffusion device 1A according to a second embodiment will be described with reference to FIG. 4. FIG. 4 is a side view showing the light diffusion device 1A according to the second embodiment. FIG. 4 is side view of the distal end part side of the light diffusion device 1A in which the structure in a tubular member 20A is also shown. In FIG. 4, the tubular member 20A is indicated by a two-dot chain line. In the following description of the second embodiment, components corresponding to those of the first embodiment are denoted by corresponding reference numerals based on the same conventions. Their descriptions may be omitted or incorporated.

The light diffusion device 1A of the present embodiment includes a laser oscillator (not shown), an optical transmission cable 10, a rod-shaped member 30, and a tubular member 20A. The light diffusion device 1A of the present embodiment mainly differs from the light diffusion device 1 of the first embodiment in the configuration of the tubular member 20A.

The tubular member 20A has an opening 22 formed on the outer periphery thereof. Specifically, the opening 22 is formed in a part of the outer periphery of the tubular member 20A that faces a refractive surface 31. With this configuration, since the tubular member 20A is not present in the optical path of laser light L emitted from an emission surface 12 via the refractive surface 31, stronger laser light L can be irradiated to the outside without being transmitted through the tubular member 20A.

### Third Embodiment

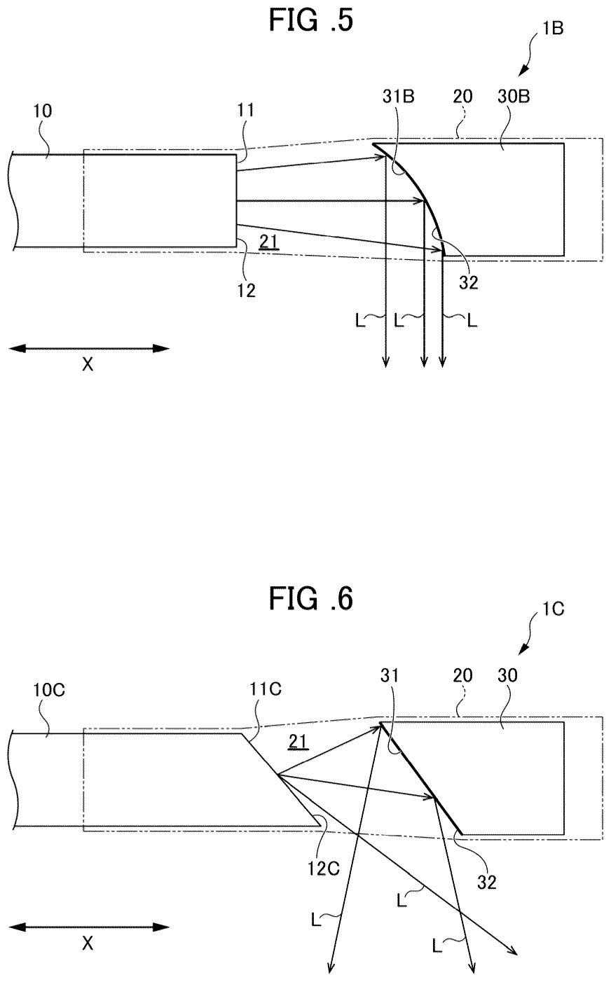

Next, a light diffusion device 1B according to a third embodiment will be described with reference to FIG. 5. FIG. 5 is a side view showing the light diffusion device 1B according to the third embodiment. FIG. 5 is a side view of the distal end part side of the light diffusion device 1B in which the structure in the tubular member 20 is also shown. In FIG. 5, the tubular member 20 is indicated by a two-dot chain line. In FIG. 5, some lines are omitted to make the figure easy to see. In the following description of the third embodiment, components corresponding to those of the first embodiment are denoted by corresponding reference numerals based on the same conventions. Their descriptions may be omitted or incorporated.

The light diffusion device 1B of the present embodiment includes a laser oscillator (not shown), an optical transmission cable 10, a rod-shaped member 30B as a reflective member, and a tubular member 20. The light diffusion device 1B of the present embodiment mainly differs from the light diffusion device 1 of the first embodiment in the configuration of the rod-shaped member 30.

The rod-shaped member 30B has a refractive surface 31B formed at an end part thereof on the optical transmission cable 10 side. The shape of the refractive surface 31B differs from that of the refractive surface 31A of the rod-shaped member 30A of the first embodiment. As shown in FIG. 5, the refractive surface 31B is formed in a curved surface shape that is concave with respect to an emission surface 12 of the optical transmission cable 10. The curvature radius of the refractive surface 31A is preferably 1200 μm. By adjusting the curvature radius of the refractive surface 31A, laser light L emitted from the emission surface 12 can be condensed as well as diffused. For example, as shown in FIG. 5, the laser light L emitted from the emission surface 12 can be uniformly emitted as a whole by the configuration of the curved refractive surface 31A that is concave with respect to the emission surface 12.

### Fourth Embodiment

Next, a light diffusion device 1C according to a fourth embodiment will be described with reference to FIG. 6. FIG. 6 is a side view showing the light diffusion device 1C according to the fourth embodiment. FIG. 6 is a side view of the distal end part side of the light diffusion device 1C in which the structure in a tubular member 20 is also shown. In FIG. 6, the tubular member 20 is indicated by a two-dot chain line. In the following description of the fourth embodiment, components corresponding to those of the first embodiment are denoted by corresponding reference numerals based on the same conventions. Their descriptions may be omitted or incorporated.

The light diffusion device 1C of the present embodiment includes a laser oscillator (not shown), an optical transmission cable 10C, a tubular member 20, and a rod-shaped member 30. The light diffusion device 1C according to the present embodiment mainly differs from the light diffusion device 1 according to the first embodiment in the configuration of a distal end part 11C of the optical transmission cable 10C.

An emission surface 12C of the optical transmission cable 10C of the present embodiment is formed by cutting the distal end part 11C obliquely with respect to the axial direction X of the optical transmission cable 10C. That is, the emission surface 12C is inclined with respect to the axial direction X of the optical transmission cable 10C. Accordingly, as shown in FIG. 6, laser light L can be emitted from the emission surface 12C while being further diffused. In the present embodiment, as shown in FIG. 6, the emission surface 12C is inclined with respect to the axial direction X of the optical transmission cable 10C so as to face a refractive surface 31 substantially in parallel. Accordingly, the optical transmission cable 10C can be brought closer to the refractive surface 31, and the laser light L transmitted through the refractive surface 31 without being refracted can be reduced

### Fifth Embodiment

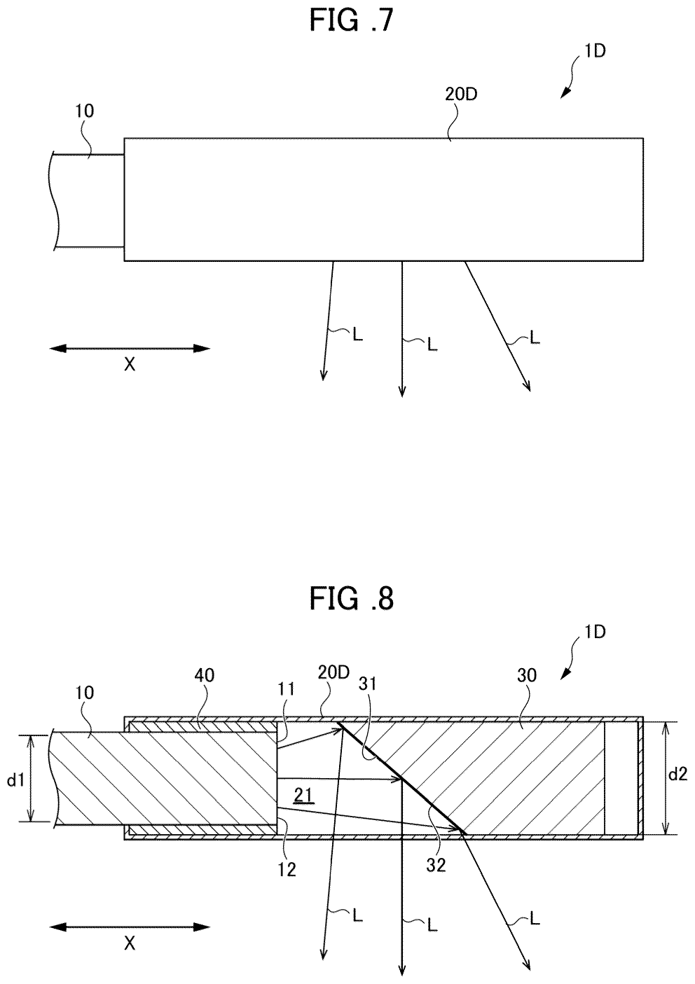

Next, a light diffusion device 1D according to a fifth embodiment will be described with reference to FIG. 7. FIG. 7 is a side view showing an appearance of the distal end part side of the light diffusion device 1D according to the fifth embodiment. FIG. 8 is a longitudinal sectional view of the distal end part side of the light diffusion device 1D in which the structure in a tubular member 20D is also shown. In the following description of the fifth embodiment, components corresponding to those of the first embodiment are denoted by corresponding reference numerals based on the same conventions. Their descriptions may be omitted or incorporated.

The light diffusion device 1D of the present embodiment includes a laser oscillator (not shown), an optical transmission cable 10, a tubular member 20D, a rod-shaped member 30, and an intervening member 40. The light diffusion device 1D of the present embodiment mainly differs from the light diffusion device 1 of the first embodiment in that it further includes the intervening member 40 and in the configuration of the tubular member 20D.

The tubular member 20D of the present embodiment is a cylindrical tube made of resin. The tubular member 20D differs from the tubular member 20 of the first embodiment in that the inner diameter thereof is slightly smaller than the outer diameter of the rod-shaped member 30 and larger than the outer diameter of the optical transmission cable 10. The rod-shaped member 30 is housed in the tubular member 20D so that the outer peripheral surface thereof and the inner peripheral surface of the tubular member 20D are in close contact with each other. On the other hand, the rod-shaped member 30 is housed in the tubular member 20D with a space between the outer peripheral surface of the rod-shaped member 30 and the inner peripheral surface of the tubular member 20D.

The intervening member 40 is made of a resin having a low refractive index. The intervening member 40 is disposed along the optical transmission cable 10 in the tubular member 20D, and fills the gap between the outer peripheral surface of the optical transmission cable 10 and the inner peripheral surface of the tubular member 20D. Examples of the resin forming the intervening member 40 include an acrylic resin. The intervening member 40 may be a layer that covers the outer peripheral surface of the optical transmission cable 10, or may be an adhesive that bonds the outer peripheral surface of the optical transmission cable 10 and the inner peripheral surface of the tubular member 20D.

According to the embodiments described above, the following effects are achieved.

The light diffusion devices 1 to 1C for photoimmunotherapy or photodynamic therapy according to the present embodiments each include the optical transmission cable 10 configured to transmit laser light L emitted from a laser oscillator and emit the transmitted laser light L from the emission surface 12 of the distal end part 11, a reflective member having the refractive surface 31 that refracts light emitted from the emission surface 12, and the tubular member 20 made of resin into which the optical transmission cable 10 and the reflective member are inserted. The refractive surface 31 is disposed at a predetermined distance from the emission surface 12 in the tubular member 20 and is inclined with respect to the axial direction X of the optical transmission cable 10. The laser light L emitted from the emission surface 12 is emitted in a direction that is inclined at a predetermined angle or more with respect to the axial direction X of the optical transmission cable 10. Accordingly, the laser light L emitted from the optical transmission cable 10 can be efficiently irradiated in a direction inclined with respect to the insertion direction of the optical transmission cable 10 via the refractive surface 31. At the time of treatment by photoimmunotherapy or photodynamic therapy, the distal end part 11 of the optical transmission cable 10 and the refractive surface 31 located on the distal end part side of the light diffusion device 1 exposed to the outside from the endoscope are disposed in the tubular member 20 made of resin. This prevents the relatively hard optical transmission cable 10 and the refractive surface 31 made of quartz from coming into contact with organs in the body, thus providing excellent biocompatibility. Further, in addition to the biocompatibility, the degree of freedom of material selection is excellent to meet the needs of device users in terms of cost, operability, etc.

In the light diffusion devices 1 to 1C according to the present embodiments, the reflective member is the rod-shaped member 30 made of quartz or silicon and is spaced apart from the optical transmission cable 10 in the tubular member 20. The refractive surface 31 is formed on an end part of the rod-shaped member 30 on the side adjacent to the optical transmission cable 10. This allows the light diffusion device 1 to be produced more easily.

In the light diffusion devices 1 to 1C according to the present embodiments, the refractive surface 31 includes a vapor-deposited metal. This allows light to be refracted more efficiently.

In the light diffusion devices 1 to 1C according to the present embodiments, the optical transmission cable 10 is a plastic fiber including a core having an outer diameter of 500 μm or more and a cladding made of resin and formed on the outer periphery of the core. The outer diameter of the refractive surface 31 viewed from the axial direction X of the optical transmission cable 10 is larger than the outer diameter of the core. Accordingly, since the outer diameter of the refractive surface 31 is larger than the outer diameter d1 of the core, it is possible to improve the tolerance for misalignment of the relative position of the refractive surface 31 with respect to the optical transmission cable 10.

In the light diffusion devices 1 to 1C according to the present embodiments, the unevenness of the refractive surface 31 on which the light is incident is equal to or less than the wavelength of the light generated from the laser oscillator. Accordingly, since the unevenness of the refractive surface 31 on which the laser light L is incident is small, it is possible to suppress heat generation by the laser light L on the refractive surface 31 at the time of irradiation.

In the light diffusion device 1C according to the present embodiment, the emission surface 12C of the optical transmission cable 10C is inclined with respect to the axial direction X of the optical transmission cable 10C. Accordingly, since the emission surface 12 of the optical transmission cable 10 is inclined obliquely, the light emitted from the optical transmission cable 10 can be further diffused.

In the light diffusion device 1C according to the present embodiment, the emission surface 12C is inclined with respect to the axial direction X of the optical transmission cable 10C so as to face the refractive surface 31 substantially in parallel. Accordingly, the emission surface 12 of the optical transmission cable 10 can be brought closer to the refractive surface 31 of the rod-shaped member 30, and the laser light L transmitted through the refractive surface 31 without being refracted can be reduced.

In the light diffusion device 1B according to the present embodiment, the refractive surface 31B is formed in a curved surface shape that is concave with respect to the emission surface 12. This allows the laser light L emitted from the emission surface 12 of the optical transmission cable 10 to be inclined by the refractive surface 31B and uniformly emitted overall.

Although the embodiments of the present invention have been described above, the present invention is not limited to the above-described embodiments and can be modified as appropriate.

### EXPLANATION OF REFERENCE NUMERALS

1, 1A, 1B, 1C, 1D light diffusion device
10, 10C optical transmission cable
11 distal end part
12 emission surface
20, 20D tubular member
30, 30B rod-shaped member (reflective member)
31, 31B refractive surface
X axial direction of optical transmission cable

1. A light diffusion device for photoimmunotherapy or photodynamic therapy, the light diffusion device comprising:
an optical transmission cable configured to transmit light emitted from a light source and emit the transmitted light from an emission surface of a distal end part;
a reflective member having a refractive surface that refracts the light emitted from the emission surface; and
a tubular member made of resin into which the optical transmission cable and the reflective member are inserted,
wherein the refractive surface is disposed at a predetermined distance from the emission surface in the tubular member and is inclined with respect to an axial direction of the optical transmission cable, and the light emitted from the emission surface is emitted in a direction that is inclined at a predetermined angle or more with respect to the axial direction of the optical transmission cable.

2. The light diffusion device according to claim 1,
wherein the reflective member is a rod-shaped member made of quartz or silicon and is spaced apart from the optical transmission cable in the tubular member, and
wherein the refractive surface is formed on an end part of the rod-shaped member on a side adjacent to the optical transmission cable.

3. The light diffusion device according to claim 1, wherein the refractive surface comprises a vapor-deposited metal.

4. The light diffusion device according to claim 1,
wherein the optical transmission cable is a plastic fiber comprising a core having an outer diameter of 500 μm or more and a cladding made of resin and formed on an outer periphery of the core, and
wherein an outer diameter of the refractive surface viewed from the axial direction of the optical transmission cable is larger than the outer diameter of the core.

5. The light diffusion device according to claim 1, wherein an unevenness of the refractive surface on which the light is incident is equal to or less than a wavelength of the light generated from the light source.

6. The light diffusion device according to claim 1, wherein the emission surface of the optical transmission cable is inclined with respect to the axial direction of the optical transmission cable.

7. The light diffusion device according to claim 6, wherein the emission surface is inclined with respect to the axial direction of the optical transmission cable so as to face the refractive surface substantially in parallel.

8. The light diffusion device according to claim 1, wherein the refractive surface is formed in a curved surface shape that is concave with respect to the emission surface.
